(19) United States
(12) Patent Application Publication
(10) Pub. No.: US 2018/0265652 A1
(43) Pub. Date: Sep. 20, 2018

(54) CROSS-LINKED BIOPOLYMER MACROSCOPIC SYSTEMS AND METHOD OF MAKING SAME

(71) Applicant: Therakine BioDelivery GmbH, Berlin (DE)

(72) Inventors: Andreas VOIGT, Berlin (DE); Sonja LEHMANN, Berlin (DE); Mariana DOBRANIS, Berlin (DE)

(21) Appl. No.: 15/517,974

(22) PCT Filed: Oct. 7, 2015
(86) PCT No.: PCT/US15/54372

§ 371 Date: Apr. 10, 2017

Related U.S. Application Data
(60) Provisional application No. 62/061,214, filed on Oct. 8, 2014.

(51) Int. Cl. C08J 3/24; A61L 27/20; A61L 27/52; A61K 8/73; A61K 8/65; A61Q 19/08

(52) U.S. Cl. CPC C08J 3/24; A61L 27/20; A61L 27/52; A61K 8/735; A61K 2800/91; A61Q 19/08; A61L 2400/06; A61L 2430/34; C08J 2305/08; A61K 8/65

(57) ABSTRACT

Disclosed are macroscopic systems of highly concentrated, cross-linked biopolymers. The macroscopic system is created by combining a biopolymer with a cross-linking agent, submerging the resulting product in an aqueous solution which contains additional cross-linking agents, to allow the biopolymer to undergo a second cross-linking process. The resulting macroscopic biopolymer has an increased biopolymer concentration and increased longevity within the body.

### CROSS-REFERENCE TO RELATED APPLICATIONS

The present application is a national stage entry according to 35 U.S.C. § 371 of PCT Application No. PCT/US2015/054372 filed on Oct. 7, 2015, which claims priority to U.S. Provisional Application Ser. No. 62/061,214 filed on Oct. 8, 2014.

### BACKGROUND

### Technical Field

The subject matter herein generally relates to macroscopic biopolymer systems. More specifically, the subject matter relates to injectable, high density, aqueous, biopolymer systems and methods for manufacturing the same.

### Background

Traditional macroscopic biopolymer systems are dissolved and cross-linked in a single step. Other traditional methods for creating macroscopic biopolymer systems include repeated addition of biopolymer and cross-linking. Such methods start with an extremely low level of biopolymer and attempt to increase the concentration at each step. The methods begin with a biopolymer system which is between a free solution and a gel system. For example, previous methods used to develop such macroscopic systems start with a hyaluronic acid system at the point between free solution (where not all the water present could be trapped by the hyaluronic acid) and a gel system (where all water is trapped by the hyaluronic acid). Such macroscopic systems have insufficient biopolymer concentration and suffer significant swelling after the cross-linking.

Macroscopic biopolymer systems are frequently used as dermal fillers and the like. The macroscopic systems are injected into the skin to help fill in facial wrinkles, improve imperfections (for example, scars), filling out lips, plumping cheeks, contouring the jaw line and other areas of the face, and restore smoother appearance to one's skin. However, the effects of such dermal fillers are only temporary. Eventually the body will absorb the filler and return to its natural state. Some biopolymer macroscopic systems currently being used as dermal fillers have an average effective time period of, for example, 3-6 months.

Therefore, there is an ongoing need in the field for an improved aqueous macroscopic biopolymer system with increased biopolymer concentration and segment density. One important goal for any new biopolymer system is limiting the volumetric increase of the biopolymer system after the cross-linking occurs; another important goal is to increase the longevity of the biopolymer system within a human or animal body.

### SUMMARY

Various embodiments are described here, and do not limit the scope in any way.

According to an embodiment, a biopolymer is partly cross-linked in a highly concentrated and hydrated configuration. The cross-linked configuration is transferred to an aqueous solution for a second cross-linking. In at least one embodiment, the resulting macroscopic system is a translucent, gelatinous composition, having an increased concentration of biopolymer. In other embodiments, the resulting macroscopic system is a translucent, liquid composition, having an increased concentration of biopolymer.

According to another embodiment, a partly cross-linked, hydrated configuration of a macroscopic biopolymer system is incubated and dissolved into an aqueous medium. A second cross-linking is conducted, connecting a dissolved biopolymer and the previously cross-linked, hydrated biopolymer, to create a macroscopic system capable of injection into a human or animal body.

According to another embodiment, a partly cross-linked, hydrated macroscopic biopolymer system is washed, dried, and micronized. The micronized, partly cross-linked biopolymer is then incubated and dissolved in an aqueous medium. A second cross-linking is conducted between the dissolved biopolymer and a hydrated, cross-linked biopolymer, creating a macroscopic system capable of injection into a human or animal body.

According to another embodiment, a partly cross-linked, hydrated configuration of a biopolymer of either microscopic or macroscopic form, is incubated and dissolved in an aqueous biopolymer solution. A second cross-linking is conducted between the dissolved biopolymer and a cross-linked biopolymer.

According to another embodiment, a hydrated and kneaded biopolymer body is taken, without cross-linking, and is used in macroscopic form.

According to another embodiment, a hydrated and kneaded biopolymer body is taken, without cross-linking, and is used in micronized form.

Those skilled in the art will recognize additional features and advantages upon reading the following detailed description, and upon viewing the accompanying drawings.

### BRIEF DESCRIPTION OF THE DRAWINGS

A representative embodiment and implementation of the present technology will now be described, by way of example only, with reference to the attached figure.

FIG. 1 shows a representative flowchart illustrating formation of a BDDE-crosslinked hyaluronic acid body.

Other representative embodiments and implementations of the present technology will now also be described.

### DETAILED DESCRIPTION

The following language and description of various embodiments are set forth in order to provide a thorough understanding of the embodiments described herein. However, it will be understood by those of ordinary skill in the art that no limitations of the present embodiments are intended, and that further alterations, modifications, and applications of the principles of the non-limiting embodiments are also included.

The term “kneaded” is defined as comprising performing repeated cycles of pressing and folding, in an algorithmic manner.

The term “micronized” is defined as having been through the process of reducing the average diameter of a solid material's particles. Any suitable micronization technique can be used in order to achieve a desired result.

The term “crosslinking” is defined as the process of chemically joining two or more polymer chains together.

The term “partially cross-linked” as used herein is intended to refer to a macroscopic biopolymer system in which fewer than all of the biopolymers are cross-linked.

The term “cross-linker” is defined as a reagent containing two or more reactive ends that are capable of attaching to specific functional groups on proteins or other molecules.

The term “hydration” refers to water that is contained within a crystalline structure of a biopolymer structure and water bound to or within a biopolymer composition.

In a non-limiting embodiment, it is an objective to get a fluid hyaluronic acid system which is flowing under a pressure difference. This system should be at its maximum hyaluronic acid concentration and still be applied via a 27 Gauge needle.

Conventional approaches use a bottom-up approach, in which more and more hyaluronic acid is dissolved and crosslinked simultaneously. With these conventional approaches, there are numerous disadvantages and drawbacks, for instance, one can potentially achieve only about 2.5% hyaluronic acid injectable solution.

According to a non-limiting embodiment, a significantly improved top-down approach is used in which the results are surprisingly advantageous. With this approach, one kneads hyaluronic acid together with a crosslinking solution (for example, BDDE, water and glacial acetic acid) (ratio hyaluronic acid to crosslinker solution: 1:1 or 1:2). Thereafter, the solid elastic body is reacting for about 4 hours at 60 degrees centigrade which is followed by a drying process (e.g. the dry body may consist or comprise of approximately 99% of crosslinked hyaluronic acid).

According to a non-limiting embodiment an objective is to obtain a system comprising a minimum number of crosslinked hyaluronic acid supramolecules but still being able to flow under a pressure difference. This surprising and unexpected observation and discovery has not been previously recognized. If all the hyaluronic acid molecules would be crosslinked into one and only one giant supramolecule the system would not flow. It has been further discovered, that if the number of crosslinked hyaluronic acid molecules is too large, or if the average number of crosslinked molecules per supramolecule is too small, the result is too close to a conventional suspension which is flowing but which is easily degraded by enzymatic attack.

It has been discovered, that what is needed is a type of “volume phase” crosslinking. The crosslinked structures should be as large and irregular as possible to let the system look like a solid elastic body if not in flow, but possessing enough thixotropy to flow under a pressure gradient.

In a non-limiting embodiment, starting with the solid matrix, which is crosslinked, one achieves significantly better control of the crosslinking itself and there is a smaller average distance between the hyaluronic acid molecules in the supramolecules.

The biopolymer of a non-limiting embodiment may be any of a wide variety of agents, which are known to those skilled in the art. Suitable biopolymers include, but are not limited to, hyaluronic acid, collagen, gelatin, albumin, hemoglobin, keratin, fibrinogen, cellulose-derivatives, biogenic carbohydrates, nucleic acids, carbon hydrate, carrageenan, pectin, alginate, chitosan, casein, whey protein, and any combination thereof. The method of manufacturing the macroscopic system may be the same for each biopolymer; however, the physicochemical properties of each biopolymer are maintained throughout the process.

The cross-linker of a non-limiting embodiment may be any of a wide variety of agents, which are known to those skilled in the art. Suitable cross-linkers include, but are not limited to, 1,4-butanediol diglycidyl either (BDDE), dimethyl suberimidate, bissulfosuccinimidyl suberate, 1-ethyl-3-[3-dimethylaminopropyl]carbodiimide hydrochloride (EDC), glutaraldehyde, formaldehyde, (succinimidyl 4[N-maleimidomethyl]cyclohexane-1 -carboxylate) (SMCC), and (sulfosuccinimidyl 4-[N-maleimidomethyl]cyclohexane-1-carboxylate) (Sulfo-SMCC). The method of manufacturing the macroscopic system may be adjusted to follow industry standard procedures for each cross-linker, so that the cross-linkers maintain their original mechanical properties.

According to a non-limiting embodiment, hyaluronic acid is partly or partially cross-linked in a highly concentrated and hydrated configuration. In a non-limiting embodiment, there is a hyaluronic acid to crosslinker solution mass ratio from about 1:2 to about 2:1.

The cross-linked configuration is transferred to an aqueous solution (for example, aqueous hyaluronic acid solution), being partially dissolved and exposed to a second cross-linking. In at least one embodiment, the resulting macroscopic system is a translucent, gelatinous composition, having an increased concentration of hyaluronic acid. In other embodiments, the resulting macroscopic system is a translucent, liquid composition, having an increased concentration of hyaluronic acid.

According to one non-limiting embodiment, a method of manufacturing a biopolymer macroscopic system comprises combining at least one first cross-linking agent with a first biopolymer to form a first partially cross-linked, hydrated biopolymer composition; and transferring the first biopolymer composition to an aqueous solution, wherein the aqueous solution comprises at least one second biopolymer and at least one second cross-linking agent, to form a second cross-linked biopolymer composition. The first biopolymer may be partially cross-linked in a highly concentrated and hydrated configuration. The biopolymer macroscopic system may include at least one biopolymer selected from the group consisting of hyaluronic acid, collagen, gelatin, albumin, hemoglobin, keratin, fibrinogen, a cellulose-derivative, a biogenic carbohydrate, a nucleic acid, carbon hydrate, carrageenan, pectin, alginate, chitosan, casein, whey protein, and any combination thereof. Each of the first cross-linking agent and the second cross-linking agent is selected from the group consisting of 1,4-butanediol diglycidyl either, dimethyl suberimidate, bissulfosuccinimidyl suberate, 1-ethyl-3-[3-dimethylaminopropyl]carbodiimide hydrochloride, glutaraldehyde, formaldehyde, (succinimidyl 4-[N-maleimidomethyl]cyclohexane-1-carboxylate), (sulfosuccinimidyl 4-[N-maleimidomethyl]cyclohexane-1-carboxylate), and any combination thereof. In one embodiment, the second cross-linked biopolymer composition, and resulting macroscopic system, is a substantially translucent, gelatinous composition, with an increased concentration of biopolymer. In another embodiment, the second cross-linked biopolymer composition, and resulting macroscopic system, is a substantially translucent, liquid composition, with an increased concentration of biopolymer.

According to another non-limiting embodiment, a method of manufacturing a biopolymer macroscopic system comprises dissolving a first partially cross-linked, hydrated biopolymer composition in an aqueous medium; and forming a second cross-linked biopolymer composition, wherein the first partially cross-linked, hydrated biopolymer composition is cross-linked with a dissolved biopolymer. The method comprises use of at least one cross-linking agent selected from the group consisting of 1,4-butanediol diglycidyl either, dimethyl suberimidate, bissulfosuccinimidyl suberate, 1-ethyl-3-[3-dimethylaminopropyl]carbodiimide hydrochloride, glutaraldehyde, formaldehyde, (succinimidyl 4-[N-maleimidomethyl]cyclohexane-1-carboxylate), (sulfosuccinimidyl 4-[N-maleimidomethyl]cyclohexane-1-carboxylate), and any combination thereof. The biopolymer macroscopic system comprises at least one biopolymer selected from the group consisting of hyaluronic acid, collagen, gelatin, albumin, hemoglobin, keratin, fibrinogen, a cellulose-derivative, a biogenic carbohydrate, a nucleic acid, carbon hydrate, carrageenan, pectin, alginate, chitosan, casein, whey protein, and any combination thereof. The macroscopic system is capable of injection into a human or animal body.

According to yet another non-limiting embodiment, a method of manufacturing a biopolymer macroscopic system, comprises washing a first partially cross-linked, hydrated biopolymer composition; drying the first partially cross-linked, hydrated biopolymer composition; micronizing the first partially cross-linked, hydrated biopolymer composition; dissolving the micronized biopolymer composition in an aqueous medium; and forming a second cross-linked biopolymer composition, wherein the dissolved biopolymer composition is cross-linked with a hydrated, cross-linked biopolymer. The macroscopic system is capable of injection into a human or animal body.

According to yet another non-limiting embodiment, a method of manufacturing a biopolymer macroscopic system comprises dissolving a first partially cross-linked, hydrated biopolymer composition in an aqueous biopolymer solution; and forming a second cross-linked biopolymer composition, wherein the dissolved biopolymer composition is further cross-linked with a cross-linked biopolymer.

The methods of manufacturing a biopolymer macroscopic system may be used to limit the volumetric increase of the biopolymer system after the cross-linking occurs; and also increase the longevity of the biopolymer system within a human or animal body.

According to non-limiting embodiment, the cross-linked hyaluronic acid configuration is a bulk phase material. As a result, the macroscopic system comprises a heterogeneous mixture of cross-linked hyaluronic acid at a micro-scale. Throughout the system, sites of low density, attained from dissolved hyaluronic acid crosslinking, and sites of high density, attained from cross-linked hydrated hyaluronic acid, exist creating a system with increased hyaluronic acid concentration and segment density.

According to another non-limiting embodiment, the increased hyaluronic acid concentration and segment density system can be composed of cross-linked hyaluronic acid components at any degree of crosslinking and size.

According to another non-limiting embodiment, micronized biopolymer particles are incubated and dissolved in an aqueous biopolymer-containing medium. A cross-linking is conducted to bond both the dissolved biopolymer and micronized biopolymer particles, creating a macroscopic system capable of injection into a human or animal body.

According to another non-limiting embodiment, the cross-linked biopolymer macroscopic system has an increased longevity in a human or animal body.

In at least one embodiment, the cross-linked biopolymer macroscopic system created is functional for use in cosmetic surgery. In another embodiment, the cross-linked biopolymer macroscopic system created is functional for use in aesthetic surgery applications. In another embodiment, the cross-linked biopolymer macroscopic system created is functional for use as a dermal filler.

In at least one embodiment, the cross-linked biopolymer macroscopic system created is functional for use in orthopedics.

In at least one embodiment, the cross-linked biopolymer macroscopic system created is functional for use in biocompatible scaffolds. In another embodiment, the cross-linked biopolymer macroscopic system created is functional for use in surgical scaffolds.

In at least one embodiment, the micronized, cross-linked particles are suspended in a macroscopic cross-linked gel, while maintaining an injectable system.

An embodiment includes a method for manufacturing a new gel configuration of cross-linked biopolymers for a macroscopic system. In at least one embodiment, cross-linking forms a macroscopic gel with about 5 percent hyaluronic acid. In this embodiment, the system can be injected through a 27 Gauge needle.

In the following, specific examples are described.

### EXAMPLE 1

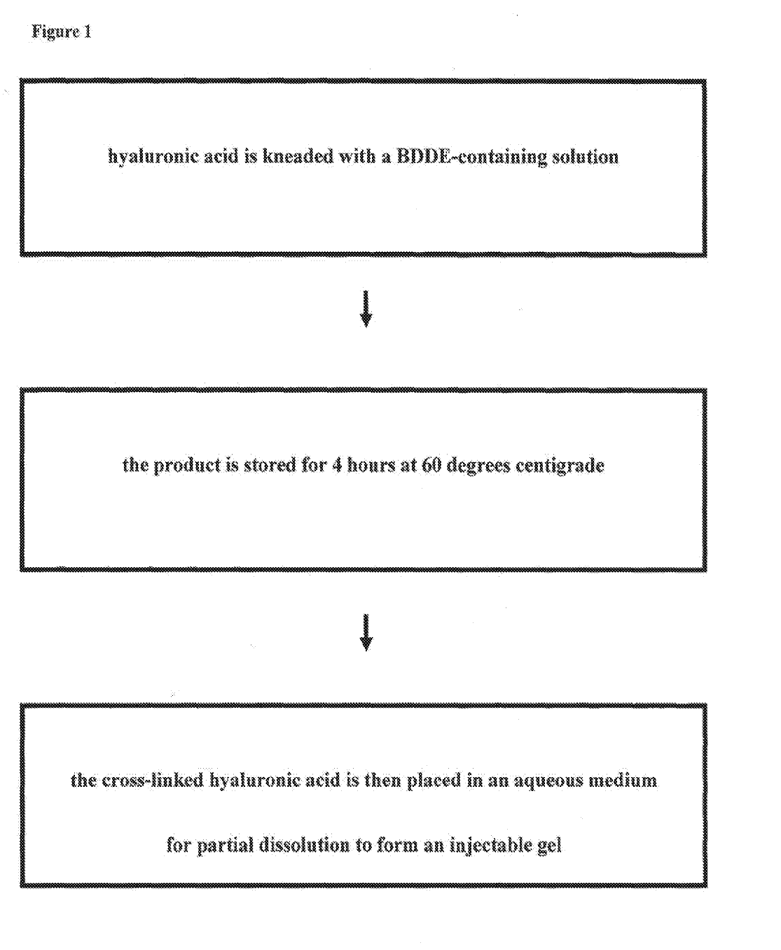

1 g of hyaluronic acid is kneaded with 2 mL of a BDDE-containing solution (ratio of BDDE to glacial acetic acid: 2:1, ratio of this mixture to water: 1:4). The product is stored for 4 hours at 60 degrees centigrade. The cross-linked hyaluronic acid is then placed in an aqueous medium for partial dissolution to form an injectable gel. FIG. 1 shows a representative flowchart illustrating formation of a BDDE-crosslinked hyaluronic acid body.

### EXAMPLE 2

Crosslinking is carried out in the same manner as in Example 1; however, hyaluronic acid is only partially cross-linked to reduce reaction time or lower concentration (but maintain the same acid pH value).

### EXAMPLE 3

In an alternative embodiment, the hyaluronic acid body may be micronized before adding it to the solution in order to obtain faster dissolution by increasing the surface area. Micronization may be carried out by a milling process at 12000 rpm. For example, one mill that may be used is a Pulverisette 14, Fritsch GmbH, Germany. However, the procedure is not limited to the Pulverisette, and may be carried out with any milling equipment that works under similar principles, or any other technology that is configured to obtain the same objective.

The process is carried out in a manner to prevent detrimental changes in temperature throughout the process (for example batch-like (less than 0.5 g) or under steady, efficient cooling conditions).

### EXAMPLE 4

Formation of an injectable gel wherein 1 g of gelatin is slowly mixed with 1 g of water and mechanically treated (kneaded) to form an elastic body. The elastic body is then dried and broken into pieces which are then milled as described in Example 3. The product is added to an aqueous solution, and a cross-linker is added to obtain a final, cross-linked, injectable gel.

### EXAMPLE 5

According to an embodiment, a biopolymer, without cross-linking (for example, hyaluronic acid, is hydrated, kneaded and is used in macroscopic form.

The embodiments shown and described herein are only examples. Even though numerous characteristics and advantages of the present technology have been set forth in the foregoing description, together with details of the structure and function of the present disclosure, the disclosure is illustrative only, and changes may be made in the detail, including in matters of shape, size and arrangement of the parts within the principles of the present disclosure up to, and including, the full extent established by the broad general meaning of the terms expressed herein.

What is claimed is:

1. A method of manufacturing a biopolymer macroscopic system, comprising:
combining at least one first cross-linking agent with a first biopolymer to form a first at least partially cross-linked, hydrated biopolymer composition; and
transferring the first biopolymer composition to an aqueous solution, wherein the aqueous solution comprises at least one second biopolymer and at least one second cross-linking agent, to form a second at least partially cross-linked biopolymer composition.

2. The method of claim 1, wherein the first biopolymer is partially cross-linked in a highly concentrated and hydrated configuration.

3. The method of claim 1, wherein the biopolymer macroscopic system comprises at least one biopolymer selected from the group consisting of hyaluronic acid, collagen, gelatin, albumin, hemoglobin, keratin, fibrinogen, a cellulose-derivative, a biogenic carbohydrate, a nucleic acid, carbon hydrate, carrageenan, pectin, alginate, chitosan, casein, whey protein, and any combination thereof.

4. The method of claim 1, wherein each of the first cross-linking agent and the second cross-linking agent is selected from the group consisting of 1,4-butanediol diglycidyl either, dimethyl suberimidate, bissulfosuccinimidyl suberate, 1-ethyl-3-[3-dimethylaminopropyl]carbodiimide hydrochloride, glutaraldehyde, formaldehyde, (succinimidyl 4-[N-maleimidomethyl]cyclohexane-1-carboxylate), (sulfosuccinimidyl 4-[N-maleimidomethyl]cyclohexane-1-carboxylate), and any combination thereof.

5. The method of claim 1, wherein the second cross-linked biopolymer composition is a translucent, gelatinous composition, with an increased concentration of biopolymer.

6. The method of claim 1, wherein the second cross-linked biopolymer composition is a translucent, liquid composition, with an increased concentration of biopolymer.

7. A method of manufacturing a biopolymer macroscopic system, comprising:
dissolving a first at least partially cross-linked, hydrated biopolymer composition in an aqueous medium; and
forming a second at least partially cross-linked biopolymer composition, wherein the first at least partially cross-linked, hydrated biopolymer composition is cross-linked with a dissolved biopolymer.

8. The method of claim 7, wherein the method comprises use of at least one cross-linking agent selected from the group consisting of 1,4-butanediol diglycidyl either, dimethyl suberimidate, bissulfosuccinimidyl suberate, 1-ethyl-3-[3-dimethylaminopropyl]carbodiimide hydrochloride, glutaraldehyde, formaldehyde, (succinimidyl 4-[N-maleimidomethyl]cyclohexane-1-carboxylate), (sulfosuccinimidyl 4-[N-maleimidomethyl]cyclohexane-1-carboxylate), and any combination thereof.

9. The method of claim 7, wherein the biopolymer macroscopic system comprises at least one biopolymer selected from the group consisting of hyaluronic acid, collagen, gelatin, albumin, hemoglobin, keratin, fibrinogen, a cellulose-derivative, a biogenic carbohydrate, a nucleic acid, carbon hydrate, carrageenan, pectin, alginate, chitosan, casein, whey protein, and any combination thereof.

10. The method of claim 7, wherein the macroscopic system is capable of injection into a human or animal body.

11. A method of manufacturing a biopolymer macroscopic system, comprising:
washing a first at least partially cross-linked, hydrated biopolymer composition;
drying the first at least partially cross-linked, hydrated biopolymer composition;
micronizing the first at least partially cross-linked, hydrated biopolymer composition;
dissolving the micronized biopolymer composition in an aqueous medium; and
forming a second at least partially cross-linked biopolymer composition, wherein the dissolved biopolymer composition is cross-linked with a hydrated, cross-linked biopolymer.

12. The method of claim 11, wherein the method comprises use of at least one cross-linking agent selected from the group consisting of 1,4-butanediol diglycidyl either, dimethyl suberimidate, bissulfosuccinimidyl suberate, 1-ethyl-3-[3-dimethylaminopropyl]carbodiimide hydrochloride, glutaraldehyde, formaldehyde, (succinimidyl 4-[N-maleimidomethyl]cyclohexane-1-carboxylate), (sulfosuccinimidyl 4-[N-maleimidomethyl]cyclohexane-1-carboxylate), and any combination thereof.

13. The method of claim 11, wherein the biopolymer macroscopic system comprises at least one biopolymer selected from the group consisting of hyaluronic acid, collagen, gelatin, albumin, hemoglobin, keratin, fibrinogen, a cellulose-derivative, a biogenic carbohydrate, a nucleic acid, carbon hydrate, carrageenan, pectin, alginate, chitosan, casein, whey protein, and any combination thereof.

14. The method of claim 11, wherein the macroscopic system is capable of injection into a human or animal body.

15. (canceled)

16. (canceled)

17. (canceled)
